(19) United States
(12) Patent Application Publication
(10) Pub. No.: US 2003/0206928 A1
(43) Pub. Date: Nov. 6, 2003

(54) BIOACTIVE, BIOABSORBABLE SURGICAL POLYETHYLENE GLYCOL AND POLYBUTYLENE TEREPHTALATE COPOLYMER COMPOSITES AND DEVICES

(76) Inventors: PERTTI TORMALA, TAMPERE (FI); MINNA KELLOMAKI, TAMPERE (FI)

Correspondence Address: KENYON & KENYON, ONE BROADWAY, NEW YORK, NY, 10004, US

(21) Appl. No.: 09/287,925

(22) Filed: Apr. 7, 1999

(51) Int. Cl. A61K 31/765; A61F 2/00

(52) U.S. Cl. 424/400

(57) ABSTRACT

The invention relates to bioactive, biocompatible, bioabsorbable surgical composites and devices, such as plates, meshes, membranes, pins, screws, tacks, bolts, intramedullary nails, suture anchors, staples, bone plugs or other devices which are applied in bone-to-bone, soft tissue-to-bone or soft tissue-to-soft tissue fixation or in guided tissue regeneration or in fixation of bioabsorbable and/or biostable implants in, and/or on, bone or soft tissue, which composites and devices are fabricated of bioabsorbable segmented block copolymer of polyethylene glycol and polybutylene terephtalate that contains bioactive ceramic particles or reinforcement fibers and optional porosity.

The invention relates to bioactive, biocompatible, bioabsorbable surgical composites and devices, such as membranes, meshes, plates, pins, screws, tacks, bolts, intramedullary nails, suture anchors, staples, bone plugs, or other devices which are applied in guided tissue regeneration or in bone-to-bone, soft tissue-to-bone or soft tissue-to-soft tissue fixation or in fixation of bioabsorbable and/or biostable implants in, and/or on, bone or soft tissue, which composites and devices are fabricated of bioabsorbable copolymers of polyethylene glycol and polybutylene terephtalate and contain bioactive ceramic particles or reinforcement fibers and optional porosity.

### BACKGROUND OF THE INVENTION

Bioabsorbable surgical devices such as, e.g., pins, screws, plates, tacks, bolts, intramedullary nails, suture anchors, or staples, etc., made from bioabsorbable polymers are becoming more frequently used in the medical profession in bone-to-bone, soft tissue-to-bone or soft tissue-to-soft tissue fixation and for guided tissue regeneration. Numerous publications describe the aforementioned and other bioabsorbable devices for such tissue management applications, e.g., U.S. Pat. No. 4,655,203, U.S. Pat. No. 4,743,257, U.S. Pat. No. 4,863,472, U.S. Pat. No. 5,084,051, U.S. Pat. No 4,968,317, EPO Pat. No. 449,867, U.S. Pat. No. 5,562,704, PCT/FI 96/00351, PCT/FI 96/00511, FI Pat. Appl. Ser. No. 965111, U.S. patent application Ser. No. 08/873,174, U.S. patent application Ser. No. 08/887,130, U.S. patent application Ser. No. 08/914,137, and U.S. patent application Ser. No. 08/921,533, the entire respective disclosures of which are incorporated herein by way of this reference.

Surgeons would prefer to use bioabsorbable devices that eventually resorb and disappear from the body after they have served their purpose during tissue fixation and/or guided tissue regeneration and healing, and accordingly, are not needed any more. However, a device made from bioabsorbable polymer must have sufficient strength and stiffness for effective tissue fixation, and it must retain sufficient strength to perform its function during the tissue healing process, before it eventually is absorbed by the body. It is advantageous to mix different additives into bioabsorbable polymers to modify their properties and to yield devices having useful properties. Such typical additives include ceramics, which optionally can be bioactive, particle fillers and short fiber reinforcements (having fiber lengths typically between 1 μm-10 mm), each of which can promote osteoconductivity of bioabsorbable fixation implants, such as pins, screws or plates or other fixation implants like suture anchors and tacks, which are in contact with bone tissue.

Bioactive, bioabsorbable ceramic fillers and fibers, and/or their use in bioabsorbable devices as bioactive ceramic fillers and/or reinforcements, have been described in several of the aforementioned publications, and also are described in, e.g., EPO Pat. Appl. 0 146 398, U.S. Pat. No. 4,612,923, and PCT Pat. Appl. WO 96/21628, the entire disclosures of each of which are incorporated herein by way of this reference.

Ceramic particle fillers and/or short fiber reinforcements typically are first dry blended with bioabsorbable polymer powder, granulate or flakes, and the mixture is then melt blended in an extruder, injection molding machine or in a compression molding machine. The melt blended extrudate can be pelletized or cooled and crushed and sieved to the desired grain size. Such pellets or grains can be further melt processed, e.g., by extrusion, injection molding or compression molding, into bioabsorbable preforms or they can be used as masterbatches and mixed with nonblended bioabsorbable polymers and melt processed into bioabsorbable preforms which can be processed further mechanically and/or thermomechanically to make surgical devices. It also is possible to melt process many devices directly from pellets or grains or masterbatches of polymer mixtures, e.g., with extrusion, injection molding or compression molding.

Particles or short fibers of bioactive glass, such as are described in PCT Pat. Appl. WO 96/21628, the entire disclosure of which is incorporated herein by way of this reference, are especially advantageous ceramic fillers and/or reinforcements in bioabsorbable polymers because they slowly dissolve under tissue conditions and form hydroxyapatite precipitations, (see, e.g., M. Brink, “Bioactive glasses with a large working range”, Doctoral Thesis, Åbo Akademi University, Turku, Finland, 1997, the entire disclosure of which is incorporated herein by way of this reference), which enhances the bone growth in contact with the surface of the device.

However, in most cases, the surface of melt-molded bioabsorbable polymer composites containing bioactive glass filler and/or fiber reinforcements is coated with a “skin” of bioabsorbable polymer which prevents the immediate direct contact of glass particles with the surrounding tissues and tissue fluids when the melt molded device has been implanted into living tissue. The advantageous direct contact of bioactive glass particles with the tissue environment can develop only weeks or months after implantation when biodegradation of the polymeric surface layer (skin) has proceeded so far that cracks or crazes have developed in the surface layer of the composite. Therefore, it is necessary to machine the surfaces of such melt molded composites mechanically to remove the isolating skin layer if immediate contact between glass particles (filler or fibers) is desired. Such surface machining is, however, a time consuming process.

An additional general problem with ceramic particle filled thermoplastic polymer composites is their brittleness, because addition of ceramic fillers into the polymer matrix changes most thermoplastic polymers from tough and ductile to brittle in nature. This is evidenced by significant reduction of both elongation at break and impact strength (see, e.g., Modern Plastics, Guide to Plastics, 1987, McGraw-Hill, New York, pp. 152-153 and Modern Plastics Encyclopedia, Mid-October Issue 1989, McGraw-Hill, New York, 1989, pp. 600, 606-607, 608-609, 614, the entire disclosures of both of which are incorporated herein by way of this reference). Moreover, even non-filled bioabsorbable thermoplastic polymer devices, which are manufactured by melt molding, may be brittle in their mechanical behavior. That brittleness can be a severe limitation on bioabsorbable devices, leading to premature breaking or to other adverse behavior (see, e.g., D. McGuire, et al., American Academy of Orthopaedic Surgeons, New Orleans, 65th Annual Meeting, Mar. 19-23, 1998, Final Program, p. 261, the entire disclosure of which is incorporated herein by way of this reference). Just as in nonbioabsorbable thermoplastic polymers, ceramic fillers also increase the brittleness of bioabsorbable polymers (see, e.g., U.S. patent application Ser. No. 09/148,838, Example 1).

Additionally, the prior art bioabsorbable, particle filled or short fiber filled composites and devices must have low porosities, because porosity weakens the composite and increases its brittleness. However, porosity provides advantages to an implant that is in contact with bone or other tissue, because (bone) tissue can grow into the pores, accelerating new tissue (bone) formation and locking the implant into contact with the tissue (bone), thereby preventing implant migration. Such surface porosity also would facilitate the contact between the growing bone and ceramic particle or fiber fillers, if the ceramic particles or fibers are at least partially exposed into the pores.

Furthermore, bioabsorbable and bioactive glasses are known to react on their surfaces. Dissolution of a glass surface starts within first hours when glass is exposed to hydrolytic conditions (in Simulated Body Fluid, SBF) which at first stage leads to formation of porous silica-gel layer on the surface of the glass. On this Silica-rich layer, calcium phosphate precipitation begins to rapidly grow leading to a continuous calcium phosphate layer on the glass surface (see FIG. 1). This process is well documented in the case of glasses, and is essential for implant bone-bonding (see e.g., M. Brink, “Bioactive glasses with a large working range”, Doctoral Thesis, Åbo Akademi University, Turku, Finland, 1997, the entire disclosure of which is incorporated herein by way of this reference; Kokubo T., “Bioactivity of glasses and glass ceramics”, in “Bone-bonding biomaterials”, eds. by P. Ducheyne, T. Kokubo, C. A. van Blitterswijk, Reed Healthcare Communications 1993 31-46, Leiderdorp, The Netherlands). However, the drawbacks of the bioactive glasses, as well as all ceramic and ceramic-like materials, are that they are hard and brittle and thus the use of glass and ceramic implants is limited. To eliminate these problems, polymer-glass composites have been developed.

However, prior art polymer composites containing bioactive glass filler exhibit the formation of bone growth promoting precipitations only after 9 weeks when a composite of biodegradable polymer matrix and bioactive glass is studied with non-exposed glass particles on the surface of composite specimens (see Niiranen H., Törmälä, P., “Bioactive glass-bioabsorbable polymer composites”, The first combined meeting, European Associations of Tissue Banks (EATB) and Musculo Skeletal Transplantation (EAMST), 10-12 September 1998, Turku, Finland p. 109), or after 2 weeks when bioactive glass is visible on the surface due to self-reinforcing process (see U.S. patent application Ser. No. 09/148,838). When a composite of biostable polymer and bioactive glass is studied, precipitations were seen after 3 days when the glass particles were uncovered by machining the surface of the device (see Marcolongo M., Ducheyne P., Cacourse W. C., “Surface reaction layer formation in vitro on a bioactive glass fibre/polymeric composite”, Journal of Biomedical Materials Research, Vol. 37, 440-448, 1997). In all of these cases, the buffer solution simulated tissue conditions and hydrolysis was done at 37° C. However, either the reaction time of the composite surface was far too long for optional enhancing of new bone formation by bioactive precipitations, or the surface was machined which is very time consuming process or in many cases where implant design is complicated, machining is impossible to perform.

It would, therefore, be advantageous to have a tough (nonbrittle), bioabsorbable composite comprising: (a) a matrix of a bioabsorbable polymer, copolymer (consisting of two or more monomer components) or polymer blend; (b) bioabsorbable, bioactive ceramic or glass particles and/or short or long fiber filler or reinforcement dispersed in the polymer matrix; which composite rapidly absorbs tissue fluids or water in hydrolytic conditions at 37° C. leading to rapidly starting dissolution of bioactive ceramic particles, spheres or fibers whereafter the dissolved ions precipitate as bioactive coating to the surface of both the polymer and the bioactive glass after a few days or hours of hydrolysis, wherein the bioactive coating starts to promote new bone formation; (c) optionally the material could also contain pores which are dispersed in the polymer matrix, wherein some free surfaces of the particles, spheres or fibers are exposed through the pores; and (d) an outer surface comprising a polymer matrix, pores and ceramic particles, spheres and/or fibers, wherein a substantial amount of the ceramic particles, spheres or fibers have at least one free surface not covered by the polymer's skin.

It would further be advantageous to have surgical implants manufactured of the composite described above, e.g., plates, membranes, meshes, pins, screws, tacks, bolts, intramedullary nails, suture anchors, staples, bone plugs, or other devices which can be applied in bone-to-bone, soft tissue-to-bone or soft tissue-to-soft tissue fixation or in guided tissue regeneration or in fixation of bioabsorbable and/or biostable implants in and/or on bone or soft tissue. It also would be advantageous to have such surgical implants manufactured of the composites described above, which implants have optional pores and bioactive ceramic particles, spheres and/or short or long reinforcement fibers (fillers) that are in direct contact with the bone or tissue to which the implant is applied.

### BRIEF SUMMARY OF THE INVENTION

The present invention is directed to surgical bioabsorbable composites and devices comprising:

(a) a tough (non-brittle) bioabsorbable polymeric matrix comprising of a segmented block copolymer of polyethylene glycol and polybutylene terephtalate, wherein the polymeric matrix is able to slowly form calcification on the surface of the device;

(b) a bioabsorbable and/or bioactive particle and/or short fiber filler or reinforcement phase dispersed in the copolymer matrix;

(c) optional pores dispersed in the polymer matrix; and

(d) an outer surface, wherein the polymer matrix, pores and particles or short fiber fillers therein are at least partially in direct contact with their environment.

According to the present invention, water is absorbed into the copolymer matrix causing swelling of the material that leads to rapid start of dissolution of bioactive glass particles, spheres and/or fibers, accompanied by precipitation of dissolved ions on the surface of copolymer matrix (calcification) which enables the enhanced bony growth to contact the copolymer matrix and bioactive glass particles.

### BRIEF DESCRIPTION OF THE DRAWINGS

FIG. 1 shows a mechanism of calcium phosphate precipitation and layer formation on the surface of bioactive glass.

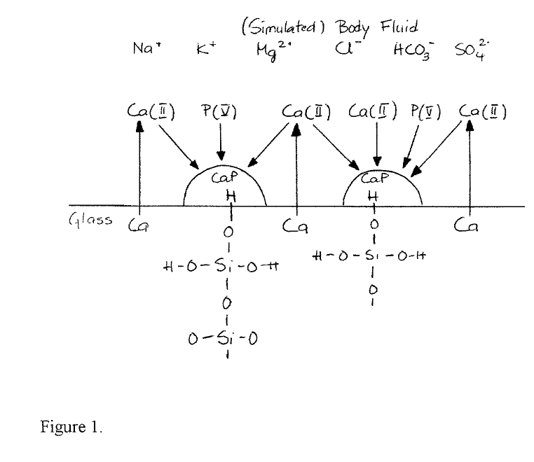

FIG. 2 is a scanning electron microscope (SEM) figure of particles of glass 13-93 (as used herein either “BG-13” or “BG 13-93,” and containing the following: Na₂O-6 wt. %; K₂O-12 wt. %; MgO-5 wt. %; CaO -20 wt. %; P₂O₅-4 wt. %; and SiO₂-53 wt-%) sieved to the particle fraction 50-125 μm.

FIG. 3(a) is a surface SEM figure of an extruded composite rod of polyethylene glycol and polybutylene terephtalate copolymer with molar ratio 70/30 containing 23±1 wt % of BG-13 glass particles, and showing totally covered and partially uncovered glass particles. The distance between scale bars (in the lower part of figure) is 1000 μm.

FIG. 3(b) is a surface SEM figure of an extruded composite rod of polyethylene glycol and polybutylene terephtalate 70/30 copolymer containing 23+1 wt % of BG-13 glass particles showing a single, partially uncovered, glass particle. The distance between scale bars (in the lower part of figure) is 100 μm.

FIG. 4 is a SEM figure of internal structure (cross section) of an extruded 1000 PEG 70/PBT 30 composite rod containing 23±1 wt % of BG-13 glass particles showing a typical single glass particle in a matrix. The scale bar is 100 μm.

FIG. 5 is a surface of glass particle on the surface of composite rod containing 23±1 wt % of BG-13 glass particles showing porous silica gel layer on the glass surface which forms when bioactive and soluble glass degrades. The composite rod was hydrolyzed 7 days in SBF. The distance between scale bars (in the lower part of figure) is 100 μm.

FIG. 6(a) is a surface of a glass particle on the surface of the composite rod containing 23±1 wt % of BG-13 glass particles showing silica gel layer with calcium phosphate precipitations on the glass surface. The composite rod was hydrolyzed 4 days in SBF. The distance between scale bars (in the lower part of figure) is 100 μm.

FIG. 6(b) is a surface of a polymer matrix close to a glass particle on the surface of the composite rod containing 23±1 wt % of BG-13 glass particles showing almost continuous calcium phosphate layer. The composite rod was hydrolyzed 4 days in SBF. The distance between scale bars (in the lower part of figure) is 100 μm.

FIG. 7 is a surface of a composite rod containing 12±1 wt % of BG-13 glass particles showing calcium phosphate layer on the glass and on the matrix (matrix is seen on the lower right hand corner). The composite rod was hydrolyzed 7 days in SBF. The distance between scale bars (in the lower part of figure) is 100 μm.

FIG. 8(a) is a surface of a glass particle on the surface of the composite rod containing 12±1 wt % of BG-13 glass particles showing silica gel layer with calcium phosphate precipitations. The composite rod was hydrolyzed 4 days in PBS. The distance between scale bars (in the lower part of figure) is 100 μm.

FIG. 8(b) shows a glass particle in between the matrix on the surface of the composite rod containing 12±1 wt % of BG-13 glass particles showing fully formed, continuous calcium phosphate layer on the matrix polymer close to the glass particle and silica gel layer with calcium phosphate precipitations and calcium phosphate layer on the glass particle. The composite rod was hydrolyzed 4 days in PBS. The distance between scale bars (in the lower part of figure) is 100 μm.

FIG. 9 is a surface of a composite rod containing 23±1 wt % of BG-13 glass particles showing fully formed calcium phosphate layer on the glass particles and on the polymer matrix. The composite rod was hydrolyzed 7 days in PBS. The distance between scale bars (in the lower part of figure) is 100 μm.

FIG. 10 is a surface of a neat polymer rod showing no changes after 7 days in vitro PBS. The scale bar is 100 μm.

FIG. 11 shows the change of volume of the rods vs. hydrolysis time.

FIG. 12 is a surface of a glass particle and matrix polymer on the surface of the composite rod containing 23±1 wt % of BG-13 glass particles showing fully formed calcium phosphate layers on both the glass and the polymer matrix surfaces. The composite rod was hydrolyzed 7 days in PBS. The distance between scale bars (in the lower part of figure) is 100 μm.

### DETAILED DESCRIPTION OF THE INVENTION

The biopolymers employed in this invention are synthetic bioabsorbable segmented block copolymers of polyethylene glycol (PEG) (which is sometimes referred also as poly ethyleneoxide, PEO) and polybutylene terephtalate (PBT). Such copolymers are disclosed in several references, e.g., in U.S. Pat. No. 5,508,036; U.S. Pat. No. 5,480,436; S Fakirov et al., Makromol. Chem., 191 (1990) 603-614; S. Fakirov et al., Makromol. Chem., 191 (1990), 615-624; D. Bakker et al., Sen-i Gakkai Symp. Preprints (1993) A33-A36; C. A. van Blitterswijk et al., in “The Bone-Biomaterial Interface” ed. by J.E. Davies. University of Toronto Press (1991) 295-307. These copolymers contain soft polyethylene glycol (PEG) blocks and hard polybutylene terephtalate (PBT) blocks in their structure. By varying the soft to hard segment ratio, and/or by varying the molecular weight of the used polyethylene glycol prepolymer, a family of copolymers is obtained in which every composition possesses a wide variety of physical and chemical properties, which are capable of inducing a wide variety of biological responses. With certain PEG/PBT ratios, and/or with certain PEG segment length, the copolymer itself promotes the calcification of polymer in vitro and is prone to bone-bonding in vivo. Generally, in both in vitro and in vivo experiments the calcification was found to occur most prominently just below the polymer surface, and not on the surface, which probably lengthens the bone-bonding reaction time. The in vivo calcification time is generally one week or longer and it is very likely that in simulating conditions the in vitro calcification takes even longer time, even though it has occurred within 4 days for 80/20 PEG/PBT in strong salt ion solution. The reported swelling of the copolymer matrix enables the transport of the necessary ions inside and out of the matrix and therefore enhances the calcification procedure. The swelling is dependent on the polyethylene glycol content, and the more PEG is in structure the more the matrix swells, and the better it also calcifies. (C. A. van Blitterswijk et al. in “The Bone-Biomaterial Interface” ed. by J.E. Davies. University of Toronto Press (1991) 295-307; P. Li et al., J Biomed. Mater. Res., 34 (1997) 79-86; C. A. van Blitterswijk et al. in “Bone-bonding biomaterials” eds. by P. Ducheyne, T. Kokubo, C. A. van Blitterswijk, Reed Healthcare Communications 1993 13-30 Laiderdorp, The Netherlands, M. Okumura et al.,in “Bone-bonding biomaterials”, eds. by P. Ducheyne, T. Kokubo, C. A. van Blitterswijk, Reed Healthcare Communications 1993 189-200, Leiderdorp, The Netherlands).

Furthermore, in cases where a bioactive glass is used as another component in the composite in addition to the PEG/PBT copolymer, swelling is an advantageous and unexpected phenomenon. The swelling expands the structure of the matrix on both a macroscopic (for example, increasing the length and diameter of the rod-shaped samples) and microscopic (i.e., molecular chains relax and start to uncoil) basis, causing the interface between the matrix and the bioactive glass to open and grow. As a result, liquids (in in vitro cases simulating buffer solutions and in in vivo cases bodily fluids) can more easily absorb into the structure of the composite along resultant channels, thereby, enabling ion exchange between liquids and composite components, facilitating easier and faster calcification.

The absorbable bioactive glasses employed in the invention can be based on P₂O₅ as the network former, such as those described in U.S. Pat. No. 4,612,923 and in prior art publications mentioned therein, the entire disclosures of each of which are incorporated herein by way of this reference. Such glasses typically can contain additionally at least one alkali or alkaline earth metal oxide, such as sodium oxide, potassium oxide, calcium oxide, magnesium oxide, and the like. Although the custom in the art is to refer to the constituents in the form of the oxides, the oxides per se need not be used in producing the glass. For instance, the following materials also can be used: (NH₄)₃PO₄, (NH₄)₂HPO₄, NaH₂PO₄, KH₂PO₄, CaCO₃, Ca(H₂PO₄)₂, MgCO₃, P₂O₅MgHPO₄, Zn₃(PO₄)₂, and MgO. As a general rule, the solubility rate (in aqueous media) is increased by increasing the proportion of alkali metal oxides (e.g., Na₂O and K₂O), and is decreased by increasing the proportion of alkaline earth metal oxides (e.g., CaO and MgO). Thus, within certain limits, the solubility rate of the glass can be varied. Other oxides also can be added, in small amounts, if desired. For example, small amounts of SiO₂, B₂O₃, and/or ZnO can be added for the purpose of retarding the dissolution rate for certain applications, or for enhancing processability.

Bioactive glasses and glass-ceramics, like those described in the Doctoral Thesis of M. Brink (see supra) and in references therein on pages 9-10, and as described by M. Marcolongo et al., J. Biomed. Mater. Res., 39 (1998) 161-170, the entire disclosure of which is incorporated herein by reference, can be employed in this invention. Naturally, the invention is not limited to those bioactive, bioabsorbable glasses described herein, but also other glasses can be used in this invention.

Suitable glasses are produced by fusing the ingredients in the desired proportions in a platinum or a dense alumina crucible. Typical fusion temperatures are 800° to 1400° C., and typical fusion times are about one to four hours. After fusion, the molten glass may be quenched, and then subjected to pulverizing to reduce the glass to a fine particle size. The pulverizing of the glass can be done by known procedures such as air jet milling, ball milling, or the like. Typically, the powders used are in the range of 1-1500 μm, preferably from 50 μm to 500 μm and most preferably from 100 μm to 300 μm. The glass can be applied also in spherical form with optimal sphere size ranges similar to those of particles. It is also within the scope of the invention to employ the glass in the form of fibers (preferably as short fibers, e.g., fibers having diameters of from about 2 to 200 microns and aspect ratios [length/diameter] of about 1 to 100). The fibers can be made by known methods such as melt spinning.

The proportion of glass filler and/or reinforcement in the polymer can vary from case to case, but will usually be within the range of from about 10 to about 60 weight percent (wt-%), based on the weight of the filled polymer. In any event, the exact proportion of glass filler is not narrowly critical. The glass is employed in an amount sufficient to increase the bioactivity of the composite.

The glass is incorporated in the polymer matrix by conventional procedures for adding fillers or short fibers to polymers. For instance, polymer pellets and glass powder or fibers, are intimately mixed in a blender, and the mixture is then compounded through an extruder. Injection or compression molding techniques can also be used. The glass can also be used in the form of continuous filaments, and rods comprising the continuous filament glass embedded in a matrix of absorbable polymer can be produced by the extrusion technique known as “pultrusion,” wherein the polymer is continuously extruded around glass filaments that are pulled through the extruder nozzle. Such composite rods can be used as such with long fibers or they can then be granulated (chopped or cut to any desired length, after the pultrusion operation) for further use in manufacturing short fiber reinforced preforms or devices by compression molding, extrusion or injection molding. Such preforms can also be oriented and/or self-reinforced with solid state deformation, like with free or die drawing, biaxial drawing, compression, hydrostatic extrusion or ram extrusion as combined with drawing. Orientation and/or self-reinforcing techniques, which can be applied to manufacture such materials, have been described in many publications, for example U.S. Pat. No. 4,968,317, EPO Pat. No. 0 423 155, EPO Pat. No. 0 442 911, FI Pat. No. 88111, FI Pat. No. 98136, U.S. patent application Ser. No. 09/036,259, U.S. Pat. No. 4,898,186, and in U.S. patent application Ser. No. 09/036,259, the entire disclosures of which are incorporated herein by way of this reference.

In this invention we have found surprisingly that by mixing into the copolymer matrix of polyethylene glycol and polybutylene terephtalate, bioactive glass particles, spheres, or short or continuous fibers it is possible to manufacture composites which:

are tough and exhibit adequate strength;

exhibit a rapid, at least partial, dissolution of bioactive glass particles or fibers in hydrolytic conditions whereafter rapid precipitation of calcium phosphate to the copolymer and glass surfaces can be seen;

have partially exposed filler particles and/or fibers on their outer surface;

swell due to water intake, which enables the rapid bony growth along the calcified surfaces of the copolymer matrix and bioactive glass; and

are optionally porous.

The new composites of the invention, when used as bone growth promoting surgical implants or as tissue growth guiding implants, or as components thereof, enhance new bone formation both in their surroundings and into the optional pores of the implant, leading to more rapid healing and new bone formation than with prior art devices.

Surgical devices made from the composites of the invention, like meshes, plates, pins, rods, intramedullary nails, screws, tacks, bolts, tissue and suture anchors, fibers, threads, cords, felts, fabrics, scaffolds, films, membranes, etc., can be applied as temporary fixation implants in bone-to-bone, soft tissue-to-bone and soft tissue-to-soft tissue fixation, and also in tissue augmentation procedures and in guided tissue regeneration.

Implants in accordance with the invention can also be reinforced additionally by fibers manufactured of a resorbable polymer or of a polymer alloy, or with other biodegradable glass fibers, or ceramic fibers, such as β-tricalciumphosphate fibers, bio-glass fibers or CaM fibers (see, e.g., EP146398).

It is natural that the materials and implants of the invention can also contain various additives for facilitating the processability of the material (e.g., stabilizers, antioxidants or plasticizers) or for changing its properties (e.g., plasticizers or ceramic powder materials or biostable fibers, such as carbon) or for facilitating its treatment (e.g., colorants). According to one advantageous embodiment of the invention, the composite also contains other bioactive agent or agents, such as antibiotics, chemotherapeutic agents, agents activating healing of wounds, growth factor(s), bone morphogenic protein(s), anticoagulants (such as heparin), etc. Such bioactive implants are particularly advantageous in clinical use, because they have, in addition to their mechanical effect and bone growth stimulating effects, other biochemical, medical and other effects to facilitate tissue healing and/or regeneration.

A typical manufacturing procedure to make devices of the present invention is as follows:

First the copolymer raw material and filler(s) and/or reinforcing fibers and optional additives in the form of a powder, flakes, pellets or granulate, etc., are homogenized by copolymer melting with a continuous process, like extrusion, or with a noncontinuous process, like injection molding or compression molding. The melted copolymer with mixed ceramics and optional additives is cooled so that it solidifies to an amorphous or partially crystalline (typically 5-50%) preform, like a cylindrical rod or bar, a flat balk with a rectangular cross-section, a plate or a sheet stock. Cooling can be done inside a mold when using injection molding or compression molding techniques. In extrusion, the preform is formed from the material melt in a die, and the preform is then passed onto a cooling belt or into a cooling solution to make a solid preform. With the used PEG/PBT molar ratios in the copolymer structure, the physical appearance of the composite material is varied from very elastomer-like to more thermoplastic-like.

Thereafter, when the copolymer matrix has such a PEG/PBT ratio that it can be formed and processed like thermoplasts, the solid preform can optionally be oriented and/or self-reinforced with an uni- and/or biaxial solid state deformation process to create an oriented preform. The self-reinforcing or orientation transforms the preform stock into a strong, tough and partially porous form. The orientation is typically made by drawing the unoriented preform in the solid state. The drawing can be done freely by fixing the ends of the preform into fixing clamps of a drawing machine, tempering the system to the desired drawing temperature, and increasing the distance between the fixing clamps so that the preform is stretched and oriented structurally. This type of orientation is mainly uniaxial. The drawing can be done also through a conical die, which can have, for example, a circular, an ellipsoidal, a square, a star-like or rectangular cross-section. When the cross-sectional area of the bioabsorbable polymer billet, which will be drawn through the die, is bigger than the cross-sectional area of the die outlet, the billet is deformed and oriented uni- and/or biaxially during drawing, depending on the geometry of billet and die.

In addition to drawing, pushing deformation can also be applied to the billet. For example, the billet may be forced through the die by drawing and at the same time by pushing the billet mechanically with a piston through the die (ram extrusion) or by pushing the billet through the die with hydrostatic pressure (see, e.g., N. Inoue, in Hydrostatic Extrusion, N. Inoue and M. Nishihara (eds.), Elsevier Applied Science Publishers, Barbing, England, 1985, p. 333-362, the entire disclosure of which is incorporated herein by way of this reference).

It also is possible to create orientation by shearing the flat billet between two flat plates which glide in relation to each other and approach each other at the same time, as is described in U.S. patent application Ser. No. 09/036,259. It also is possible to deform the billet in a compression molding device between flat plates that are pushed towards each other so that the billet deforms biaxially between the plates and attains the desired final thickness. The deformation can be done also by rolling the rod-like or plate-like preform between rollers, which flatten the preform to the desired thickness orienting the material at the same time biaxially. The rolling can be combined with drawing, e.g., by using two pairs of rollers positioned one pair after the other, which rollers have different rolling speeds. The billet and/or die, compression plates or rolls can be heated to the desired deformation temperature with electrical heating or with a suitable heating medium, like a gas or heating liquid. The heating can be done also with microwaves or ultrasonically to accelerate the heating of the billet. Regardless of the deformation method, the purpose of the solid state deformation is the orientation of the material uni- and/or biaxially so that the material is transformed into a strong and ductile one, and porosity is created around the filler and/or reinforcement particles, spheres or fibers, thus enhancing the interaction of filler and/or reinforcement with its environment.

Surgical devices can be formed from the extruded, injection molded or optionally oriented preforms by machining, stamping, thermoforming or with other mechanical, thermal or thermomechanical methods. After finishing, cleaning and drying, the surgical devices of the invention can be packed into a plastic foil and/or aluminum foil pouches which are sealed. Another drying step and filling of the pouch with an inert gas (like nitrogen or argon gas), before heat sealing of the pouch, may also be carried out.

In the next step the devices closed into the packages, are sterilized with γ-radiation, using a standard dose of radiation (e.g., 2.5-3.5 Mrad). If gas sterilization (like ethylene oxide) or plasma sterilization, will be used, the devices must be sterilized before closing the package.

Naturally, the above-mentioned steps of manufacturing devices of the present invention may further include additional steps, such as for quality control purposes. These additional steps may include visual or other types of inspections during or between the various steps, as well as final product inspection including chemical and/or physical testing and characterization steps, as well as other quality control testing.

The following examples describe some important embodiments of the invention.

### EXAMPLE 1

Manufacturing of Bioactive Glass 13-93

Bioactive glass 13-93 was manufactured according to PCT Pat. Appl. WO 96/21628, the entire disclosure of which is incorporated herein by way of this reference.

Raw materials (Na₂CO₃, CaCO₃, CaHPO₄*2H₂O, SiO₂, MgO, K₂CO₃) were measured as powders, mixed and melted in a platinum crucible at 1360° C. for 3+3 hours to form bulk glass. Bulk glass was then used for manufacturing particles, spherical particles and fibers.

Glass Particles

Bulk glass was crushed in an agate (99.9% SiO₂) grinding bowl with agate grinding balls in a planetary mill (Fritch Pulverisette 5, Germany). Agate bowl and balls were used to avoid glass contamination during grinding.

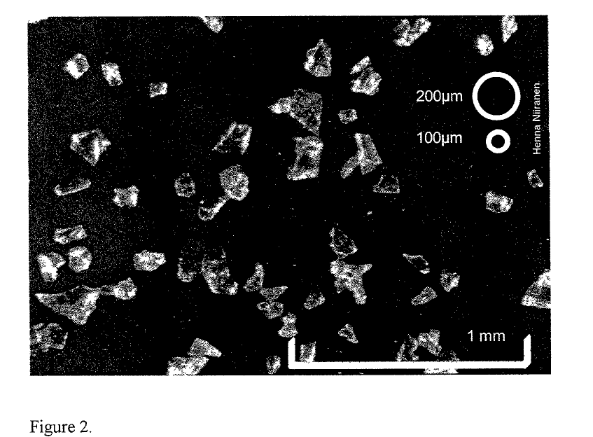

Particles (see FIG. 2) were sieved to the particle fraction 50-125 μm and washed with ethanol.

Fiber Spinning

The continuous glass fibers were manufactured by a melt spinning (drawing) process using bioactive glass 13-93.

Glass particles were heated in a platinum crucible to the temperature where the viscosity range for fiber drawing is achieved (<1000° C., about 30-60 min). A platinum crucible with 4 orifices, approximate diameter 3.6 mm, at the bottom was used. The viscous glass melt formed drops at the crucible orifices. When the drops started to fall they were caught/touched and pulled to form the fibers and attached to the take-up wheel. By varying the spinning velocity the fiber diameter could be modified.

Glass fibers with diameters of about 63 μm and 113 μm were manufactured and their tensile strength and modulus were determined.

The fibers (ten specimens) were tested just after fiber spinning in air at room temperature with a tensile testing machine (Instron 4411, Instron Ltd, England) at a cross head speed of 20 mm/min (standard recommendation: ASTM D 3379-75, Standard Test Method for Young's Modulus for High-Modulus Single-Filament Materials). TABLE 1 below provides some fiber tensile strength and modulus values as recorded.

TABLE 1

| | Average | | | |
|---|---|---|---|---|
| Average | tensile | | | |
| diameter | strength | Standard | Modulus | Standard |
| (μm) | (MPa) | deviation | (GPa) | deviation |
| 63 | 849 | 204 | 43.2 | 10.2 |
| 113 | 727 | 214 | 44.4 | 7.5 |

### EXAMPLE 2

Manufacturing of Composites of a Copolymer of Plyethylene Glycol and Polybutylene Terephtalate and Bioactive Glass (BG) 13-93 Particles

Manufacturing of Composite Rods

Polyethylene glycol and polybutylene terephtalate copolymer powder, with a molar ratio of 70/30 and PEG segment length of 1000 Da with different weight fractions (from 0 wt. % to 30 wt. %), and the glass particles of EXAMPLE 1 were mixed mechanically and poured into a hopper of a single screw extruder (model Gimac TR ø 12/24 B.V.O, of MAC.GI SRL, Castronno, Italy). A nitrogen atmosphere (N₂ flow 5 l/min) was supplied to the hopper to avoid contact with the room's air. The rotating screw, together with friction of compression and heating of the outside of the extruder barrel, plasticized the thermoplastic material and pushed the polymer melt-glass powder mixture towards the barrel end and the orifice. Temperatures of the heating zones (from feed zone to the orifice) were 120° C.-130° C.-140° C.-145° C.-147° C. and 152° C. (at the orifice).

The cylindrical extrudate rods with diameters of 2-8 mm were precooled in a N₂ atmosphere and placed on a transportation belt for cooling to room temperature. Mechanical tests (shear) (see Manninen M. J., Pohjonen T., “Intramedullary nailing of the cortical bone osteotomies in rabbits with self-reinforced poly-L-lactide rods manufactured by fibrillation method”, Biomaterials Vol. 14 (1993) no. 4, pp. 305-312) were done at room temperature for extruded and γ-sterilized rods (diameter of 3.0 mm) with different weight fractions of bioactive glass particles (using the testing machine designated Instron 4411, available from Instron Ltd, England). The rods were tested dry. Shear strength decreased from 8.75 MPa to 7.11 MPa when the portion of glass particles increased from 0 wt. % to 23 wt-%. The strengths were typical for elastomer-like polymers.

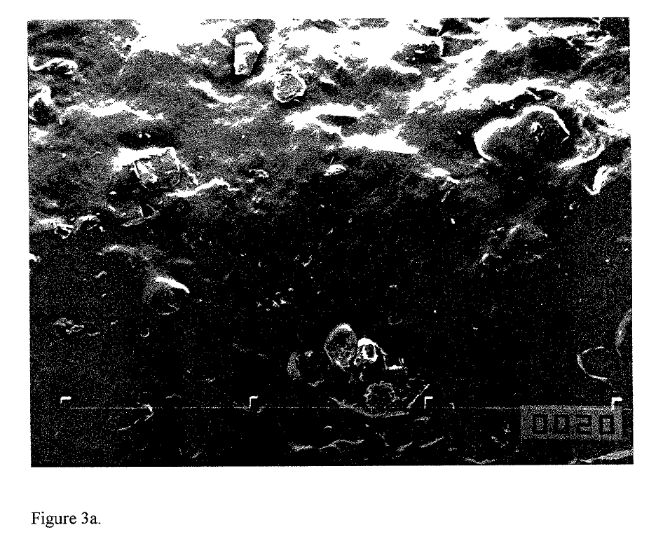

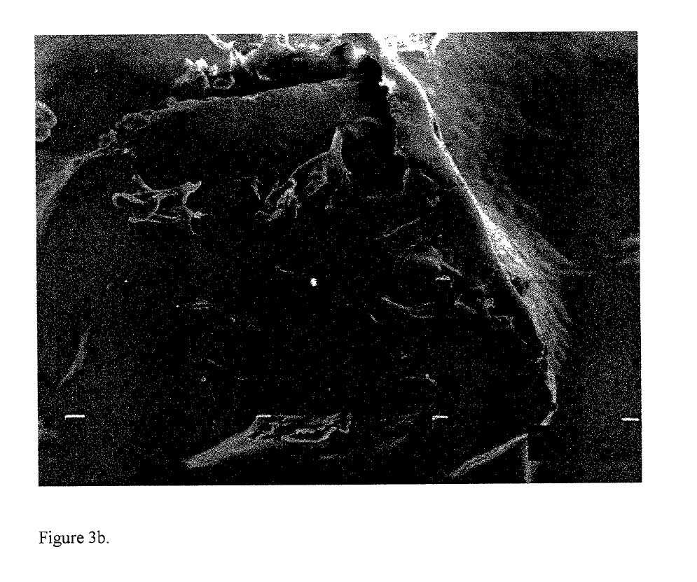

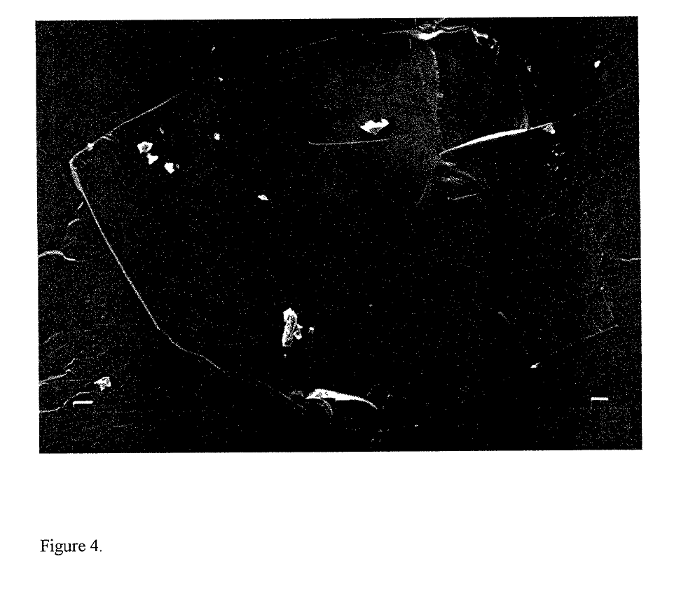

FIGS. 3(a) and 3(b) show SEM micrographs of a surface of an extruded composite rod with 23±1 wt. % of glass particles of EXAMPLE 1. Glass particles can be seen clearly below the polymer surface (skin) and some of the particles have remained partially uncovered due to the elastomeric feature of the used copolymer. FIG. 4 shows an SEM micrograph of a cross section of an extruded composite rod with 23+1 wt. % of glass particles of EXAMPLE 1. The liquid N₂-cooled rod was bent cut, and the exposed internal structure was studied by SEM. The glass particle is particularly well attached to the matrix with maximum 1 μm gap in between the copolymer matrix and the filler glass.

### EXAMPLE 3

Hydrolysis of Bioactive Copolymer-Bioactive Glass Composites

In hydrolytic conditions such as simulated body fluid (SBF), bioactive glasses dissolve partially (starting from the glass surface) leading to a formation of a silica-rich layer with further calcium phosphate or carbonated hydroxyapatite layer precipitation on the glass surface (see, e.g., M. Brink “Bioactive Glasses with a Large Working Range” Doctoral Thesis Abo Akademi University, Turku, Finland, 1997, and M. Marcolongo et al. J. Biomed. Mater. Res. 39 (1998) 161, the entire disclosures of each of which are incorporated herein by way of this reference). The formation of such precipitations is an indication of bioactive behavior of the bioabsorbable composite, and such precipitations are advantageous especially in bone surgery because they enhance new bone growth in close contact with the implant surface.

In this example, the bioactive behavior of the materials of the invention were studied in comparison to the behavior of prior art materials by examining the degradation of polymeric and composite samples in simulated body fluid (SBF) (see T. Kokubo et al. in Bioceramics, Vol. 2, ed. G. Heimke, Deutsche Keramische Gesellschaft e.V., Cologne, Germany, 1990 pp. 235-242, the entire disclosure of which is incorporated herein by way of this reference), and in phosphate buffer saline (PBS) with composition of 154 mM Na⁺, 101 mM Cl⁻, 24 mM HPO₄²⁻and 5 mM H₂PO_{hu-.}

Cylindrical samples (diameter 3 mm and length 15 mm) were placed into plastic pots filled with 200 ml of SBF or PBS. Sample solutions were kept at 37° C. for one week. The reaction on the surface and inside of the cylindrical samples were examined from dried and gold coated sample surfaces using SEM. The internal structure was studied from the specimens that were cut-and-bent at the ambient temperature exposing the inside structure of the rods.

The following samples were examined:

(A) Extruded polyethylene glycol and polybutylene terephtalate copolymer (with PEG/PBT molar ratio 70/30 and PEG segment length of 1000 Da) rod;

(B) Hydrolysis in SBF: Extruded composite rod of polyethylene glycol and polybutylene terephtalate copolymer (with PEG/PBT molar ratio 70/30 and PEG segment length of 1000 Da) with 23 wt. % of glass BG-13 particles; and

(C) Hydrolysis in PBS: Extruded composite rod of polyethylene glycol and polybutylene terephtalate copolymer (with PEG/PBT molar ratio 70/30 and PEG segment length of 1000 Da) with 23 wt % of glass BG-13 particles.

Rod surface reactions were examined with SEM 2, 4 and 7 days after immersion of the samples in buffer solutions. The results are given below in TABLE 4.

TABLE 4

| Sample | 2 days | 4 days | 7 days |
|---|---|---|---|
| A | No significant | No significant | No significant |
| | changes | changes | changes |
| B | Bioactive surface | Bioactive surface | Continuous calcium |
| | formation (1) | formation (1) | phosphate layer (2) |
| C | n/a | Bioactive surface | Continuous calcium |
| | | formation (1) | phosphate layer (2) |

(1) A porous silica gel layer forms on the surface of the glass particles on which a calcium phosphate precipitation begins to rapidly grow. Furthermore, simultaneously on the polymer matrix surfaces close to glass particles, calcium phosphate precipitations also forms, and eventually becomes so dense that continuous calcium phosphate layer covers polymer matrix surface close to glass particles. (2) The calcium phosphate precipitations on the glass particles has formed a fully developed, continuous calcium phosphate layer. In addition, the calcium phosphate precipitations and continuous calcium phosphate layers on the surface of the polymer matrix are spreading to the areas further away from glass particles.

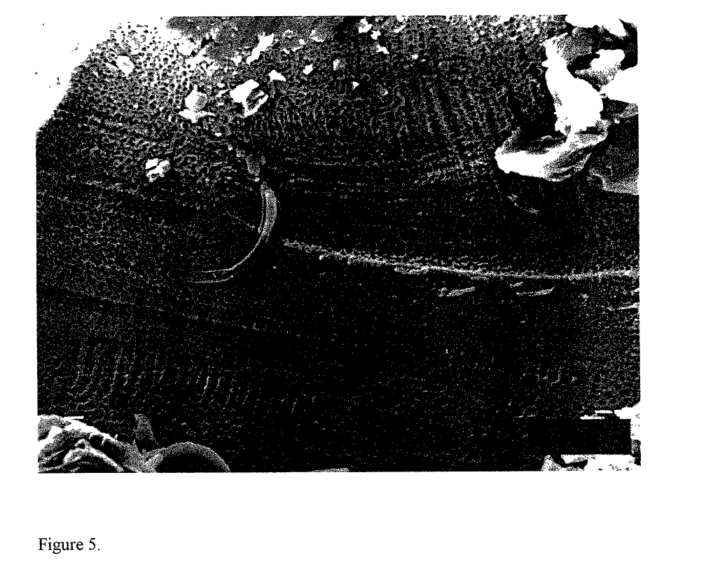

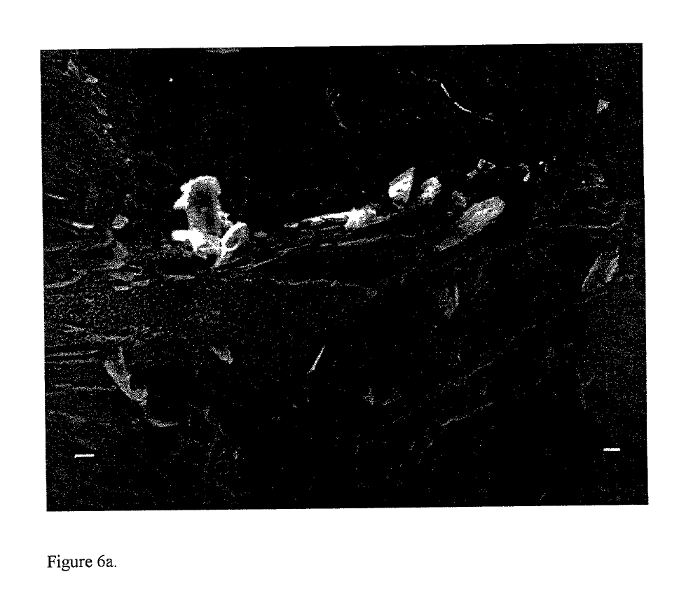

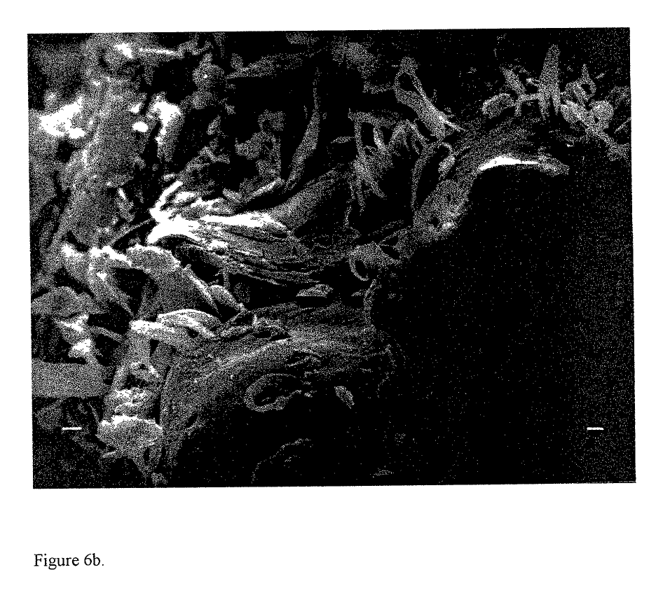

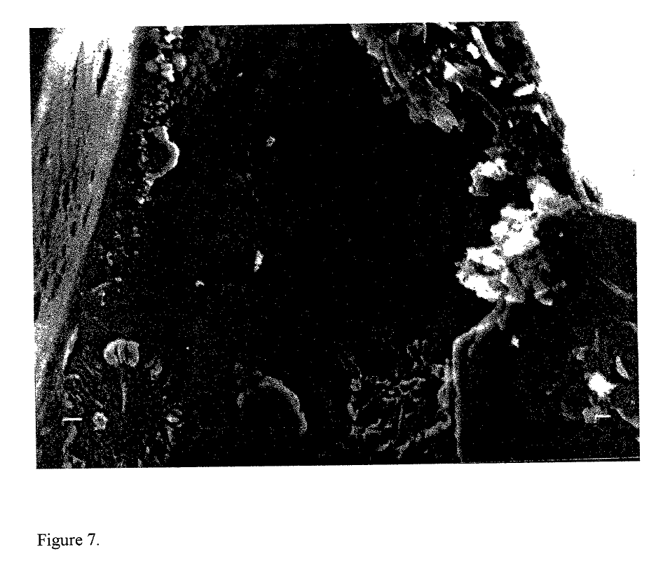

FIG. 5 provides an example of the type (1) behavior discussed above showing a surface of a glass particle on the surface of a composite rod where a silica gel layer has formed in vitro after 2 days in SBF. Likewise, FIGS. 6(a) and 6(b) also exhibit type (1) behavior, where at 4 days in SBF, the silica gel layer is prominent on the surface of the glass (FIG. 6(a)), and on the matrix surface close to bioactive glass particles where a continuing calcium phosphate layer appears (FIG. 6(b)). FIG. 7 provides an example of a type (2) behavior, where at 7 days in SBF, a continuous calcium phosphate layer is formed on both the polymer matrix surface and on the glass particle surface on the surface of a composite rod.

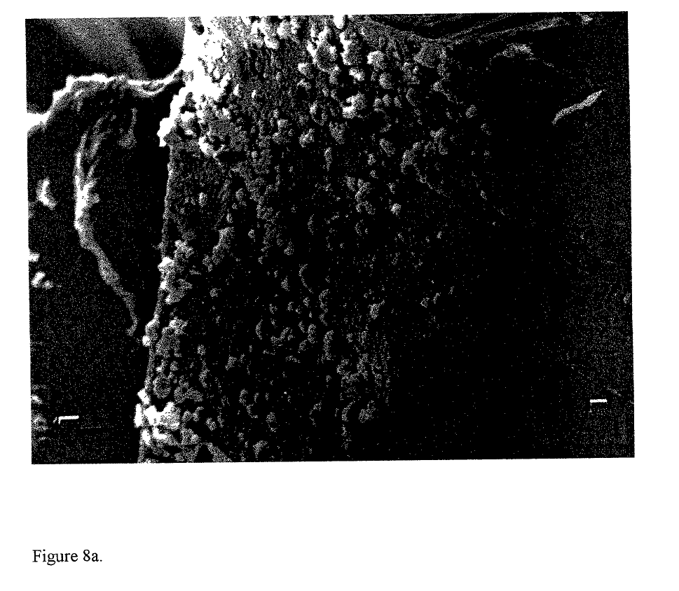

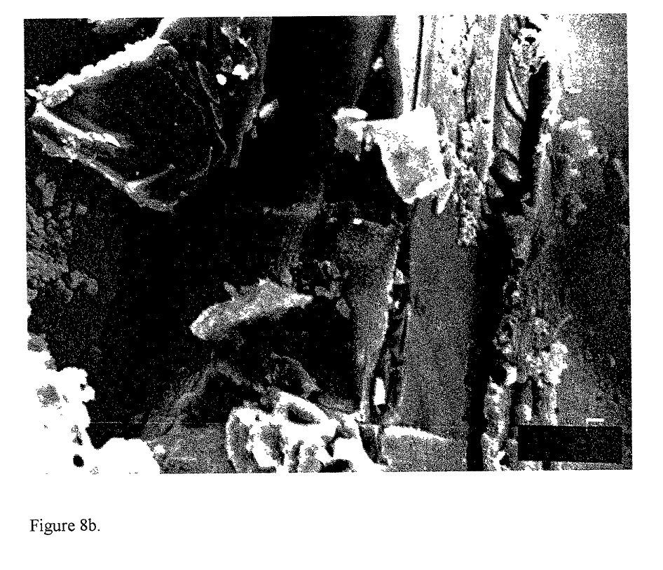

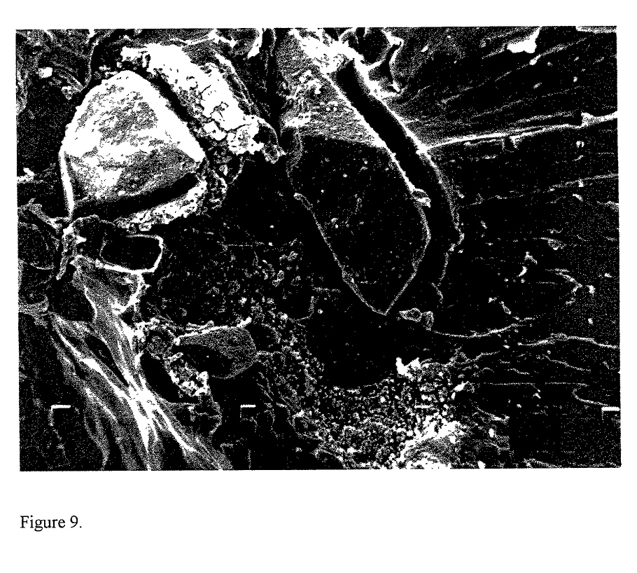

In comparison, FIGS. 8(a) and 8(b) again exhibit a type (1) behavior where at 4 days in vitro in PBS the silica gel layers and the calcium phosphate precipitations are seen on the glass particles and matrix surface. FIG. 9, on the other hand, shows that at 7 days in PBS a continuous calcium phosphate layer has formed on the glass surface, and the layer is spreading over a larger area of the matrix surface (a type (2) behavior).

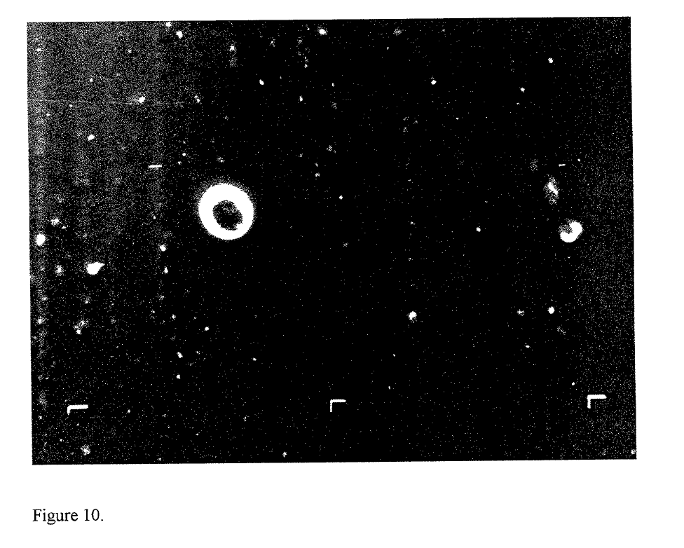

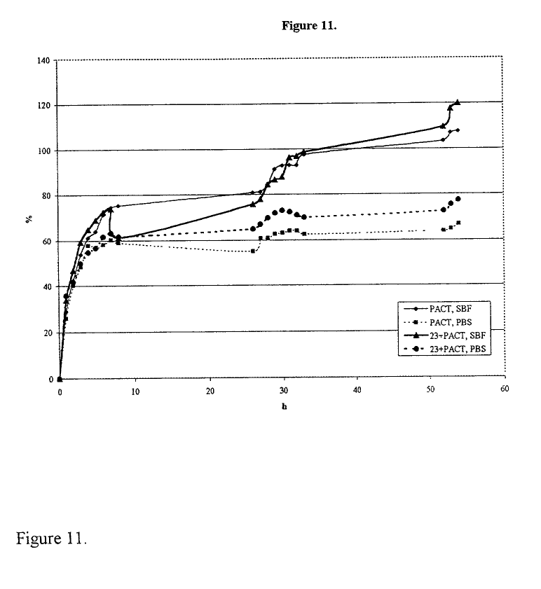

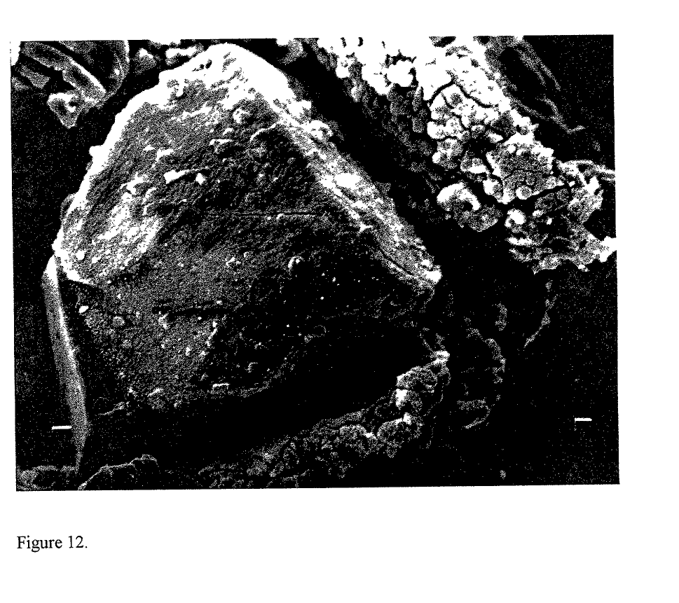

At 7 days, no changes were seen on the surface of the neat polymer rod (FIG. 10). However, the rods showed swelling according to FIG. 11, where the swelling occurred in both the polymer matrix and glass particles. Furthermore, the interfacial gap in between these two phases expanded, exposing more glass and matrix surface prone to calcification (compare to FIGS. 3(b), 4 and 12).

The internal structure of the rods was studied using SEM at the same time intervals as the surfaces. Similar types of behavior as those seen on the surface of the composite rods were observed with the internal structure. However, the reactions on the internal structure occurred at a delayed time interval. The results for the internal structure are given below in Table 5.

TABLE 5

| Sample | 2 days | 4 days | 7 days |
|---|---|---|---|
| A | No | No | No |
| | significant | significant | significant |
| | changes | changes | changes |
| B | No | Bioactive | Continuous |
| | significant | surface | calcium |
| | changes | formation (1) | phosphate |
| | | | layer (2) |
| C | No | Bioactive | Continuous |
| | significant | surface | calcium |
| | changes | formation (1) | phosphate |
| | | | layer (2) |

This example demonstrated that bioabsorbable copolymer rods with bioactive glass particles (samples B and C), exhibited surprisingly bioactive behavior already after 2 to 4 days of hydrolysis in the SBF, and 4 to 7 days in the PBS. This is a much more rapid bioactive behavior than the prior art polymer-bioactive glass composites with non-machined or non-treated surfaces (see, e.g., U.S. patent application Ser. No. 09/148,838 and Niiranen H., Törmälä, P., “Bioactive glass-bioabsorbable polymer composites”, The first combined meeting, European Associations of Tissue Banks (EATB) and Musculo Skeletal Transplantation (EAMST), 10-12 September 1998, Turku, Finland p. 109).

### EXAMPLE 4

Glass fibers (with diameter 113 μm) of EXAMPLE 1 were coated with copolymer of polyethylene glycol and polybutylene terephtalate, with a PEG/PBT molar ratio of 70/30 and polyethylene glycol segment length of 1000 Da, by drawing a bundle of 20 continuous fibers through the polymer melt, and cooling the polymer-impregnated fiber bundle in air. The amount of glass fibers was 50 wt. % in the impregnated bundle. The bundle was cut to 3 mm long granules and these were mixed mechanically with pure copolymer of polyethylene glycol and polybutylene terephtalate (PEG/PBT molar ratio of 70/30 and polyethylene glycol segment length of 1000 Da) powder so that the amount of glass fibers was 25 wt. % in the mixture. The mixture was melt extruded into rods with diameter of 3 mm.

SEM examination of rod surfaces and the inner structure showed that glass fibers had broken during extrusion to the lengths mainly between 150 μm-1.5 mm. The fibers were partially oriented with their long axes in the extrusion direction. Bioactivity of extruded rods (diam. 3 mm, length 20 mm) was studied in vitro in simulated body fluid (SBF) and in phosphate buffer saline (PBS) according to EXAMPLE 3. After 4 days immersion of samples in SBF and 7 days in PBS, continuous calcium phosphate precipitations were seen on surfaces of rods on both the bioactive glass fibers and the copolymer matrix close to glass fibers while the corresponding copolymer rods (without any bioactive glass additive) showed no changes at that time scale. Thus, this example demonstrated that bioabsorbable copolymer rods with bioactive glass fibers, surprisingly exhibited bioactive behavior already after 2 to 7 days hydrolysis. This is partly because the glass fibers remained partially uncovered at extrusion, allowing the buffer solution to effect the glass immediately in hydrolytic conditions without the need for a preliminary processing stage, such as machining or solid state drawing, to expose glass particles.

What is claimed is:

1. A bioactive, bioabsorbable surgical composite material or device made therefrom, comprising:
a bioabsorbable segmented block copolymer matrix having a surface and comprised of polyethylene glycol and polybutylene terephtalate, and having bioabsorbable and bioactive particles, spheres or fibers dispersed into the matrix, said particles, spheres or fibers being comprised of glass or ceramic, wherein said particles, spheres or fibers are at least partially exposed on the surface of the matrix, and wherein said particles, spheres or fibers dissolve and create bioactive precipitation on the surface of the matrix no later than 12 days after said matrix is inserted in vivo.

2. A composite material or device according to claim 1, wherein the bioactive precipitation on the surface of the matrix forms no later than 7 days after the matrix is inserted in vivo.

3. A composite material or device according to claim 1, wherein the bioactive precipitation on the surface of the matrix forms no later than 2 to 4 days after the matrix is inserted in vivo.

4. A composite material or device according to claim 1, wherein the bioactive precipitation on the surface of the matrix forms no later than 2 to 4 hours after the matrix is inserted in vivo.

5. A composite material or device according to claim 1, wherein the bioactive precipitation forms as a result of the swelling of the composite material or device when exposed to hydrolytic conditions.

6. A composite material or device according to claim 5, wherein the swelling of the material or device leads to the opening of the structure of the copolymer matrix at the interface of said matrix with the particles or fibers.

7. A composite material or device according to claim 1, wherein the matrix is porous.

8. A composite material or device according to claim 1, wherein the material or device is oriented or self-reinforced.

9. A composite material or device according to claim 1, wherein the material or device contains an alkali or alkaline earth metal.

10. A composite material or device according to claim 9, wherein the alkali or alkaline earth metal are in the form of oxides.

11. A composite material or device according to claim 1, wherein the material or device is capable of enhancing calcification behavior in bone to which the device or material is attached.
